(19) United States
(12) Patent Application Publication
(10) Pub. No.: US 2013/0110123 A1
(43) Pub. Date: May 2, 2013

(54) DEVICES AND METHODS TO POSITION AN ANCHOR DURING A SURGICAL PROCEDURE

(71) Applicants: David Foirella, Memphis, TN (US); John Stewart Young, Olive Branch, MS (US)

(72) Inventors: David Foirella, Memphis, TN (US); John Stewart Young, Olive Branch, MS (US)

(73) Assignee: WARSAW ORTHOPEDIC, INC., Warsaw, IN (US)

(21) Appl. No.: 13/282,238

(22) Filed: Oct. 26, 2011

(51) Int. Cl. A61B 17/88

(52) U.S. Cl. 606/104

(57) ABSTRACT

Devices and methods for positioning an anchor within a patient during a surgical procedure. The devices are designed to interface with an anchor. The interface facilitates attachment of instruments to an exterior of the anchor. The devices may further be sized for use in minimally invasive surgical procedures where direct visualization and manipulation of the anchor is often impractical. The device may include first and second members that are movable relative to each other. The members include distal ends sized for insertion into a head of an anchor. The device may include a handle operatively connected to one or both members to move the members relative to each other to expand a size of the first member to engage with the head. The engagement is confined with the exterior of the head remaining exposed for attachment of an instrument to the head. One or both members may further be shaped to facilitate attachment of the instrument to the head.

### BACKGROUND

The present application is directed to devices and methods for positioning an anchor within a patient and, more specifically, to devices and methods for engaging an anchor head and positioning the anchor during a surgical procedure.

Surgical anchors are used for attaching an elongated member such as a rod, tether, or wire to bone or tissue within a patient. One type of anchor includes a shaft that attaches to the bone or tissue, and a head that is movably attached to an end of the shaft. The head includes a channel sized to receive the elongated member. In use, the anchor is inserted into a patient with the shaft being attached to the bone or tissue and the head positioned outward beyond the bone or tissue. The head is then moved relative to the shaft to orient the channel in a position to receive the elongated member.

It is often difficult to maintain the head at the desired angular orientation relative to the shaft after the anchor is inserted into the patient and prior to insertion of the elongated member. The head may be freely movable relative to the shaft and therefore may move from the desired orientation if it is released by the medical personal prior to insertion of the elongated member. This requires the medical personal to maintain contact with the head to maintain the desired orientation.

Various types of tools may be used to hold the head at the desired orientation prior to insertion of the elongated member into the channel. However, these tools contact against an exterior of the screw head. This arrangement against the exterior may prevent attaching an instrument to the anchors that may be necessary for the surgical procedure.

Therefore, there is a need for a device for positioning the head of the anchor that has been inserted into a patient.

### SUMMARY

The present application is directed to methods and devices for positioning an anchor within a patient. One device includes elongated first and second members that are movably connected together for movement of the first member relative to the second member. Each of the members includes a distal end and a proximal end. The device also includes a collet section at a distal end of the first member. The collet section includes a slot that that extends inward from the distal end and longitudinally divides the first member into a first portion on a first side of the slot and a second portion on an opposing second side of the slot. The second member includes a base at the distal end. The base includes opposing first and second angled surfaces that form a peak with the base positioned such that the peak is aligned with the slot in the collet section. The first member is movable towards the second member to separate the collet section at the slot with the first portion configured to move along the first angled surface and move outward in a first lateral direction and the second portion configured to move along the second angled surface and move outward in a second lateral direction.

Another device includes a first member having an elongated substantially straight first section, a first bent section that bends away from a longitudinal axis of the straight first section, and a first distal end on an opposing side of the first bent section from the straight first section. The device also includes a second member having an elongated substantially straight second section, a second bent section that bends away from a longitudinal axis of the straight second section, and a second distal end on an opposing side of the second bent section from the straight second section. The second member is positioned relative to the first member with the second distal end being positioned longitudinally beyond the first distal end. A connection connects together the first and second members with the first and second sections being aligned. A slot in the first member extends inward from the first distal end and divides the first member into a first portion on a first side of the slot and a second portion on a second side of the slot. A base at the second distal end includes a first side that faces towards the first distal end and includes a pair of ramped surfaces, and an opposing second side configured to seat within the anchor. The first member is movable relative to the second member between a first orientation with the first distal end having a first width and a second orientation with the first distal end overlapping the base to separate the first and second portions a greater amount with the first distal end having a larger second width.

The application also includes methods of positioning an anchor within a patient during a surgical procedure. One method includes positioning a collet section of an elongated first member and a base of an elongated second member in a channel in a head of an anchor with the base at a bottom of the channel and the collet section above the base and away from the bottom. The collet section includes first and second longitudinal portions separated by a longitudinal slot that extends inward from a distal end of the first member. The base includes a first side that faces towards the collet section with first and second ramped surfaces that form a peak that increases in width away from the collet section. The method includes moving the collet section relative to the base towards the bottom of the channel, and moving the first portion of the collet section along the first ramped surface of the base and moving the second portion of the collet section along the second ramped surface of the base. This movement increases a width of the collet section as the first and second portions move along the ramped surfaces. The method also includes engaging outer surfaces of the first and second portions with the anchor head that forms the channel with an exterior of the anchor head being exposed to attach with an instrument.

The various aspects of the various embodiments may be used alone or in any combination, as is desired.

### BRIEF DESCRIPTION OF THE DRAWINGS

FIG. 1 is a schematic view of a device positioned at an anchor and an extender configured to attach to the anchor.

FIG. 2 is a perspective view of a device.

FIG. 3 is a perspective view of a distal ends of first and second members of a device.

FIG. 4 is a perspective view of the distal ends of the device of FIG. 3 positioned relative to an anchor.

FIG. 5 is a top perspective view of apertures in first and second members of the device that align to receive a guide wire.

FIG. 6 is a side view of distal sections of first and second members.

FIG. 7 is a side view of distal sections of the first and second members positioned relative to an anchor.

FIG. 8 is a perspective view of a device attached to an anchor with an extender instrument also attached to the anchor.

FIG. 9 is a side view of distal sections of first and second members.

FIG. 10 is a side view of distal sections of the first and second members.

### DETAILED DESCRIPTION

The present application is directed to devices and methods for positioning an anchor within a patient during a surgical procedure. The devices are design to interface with the anchor in a manner that facilitates attachment of instruments to an exterior of the anchor. The devices may further be sized for use in minimally invasive surgical procedures where direct visualization and manipulation of the anchor is often impractical.

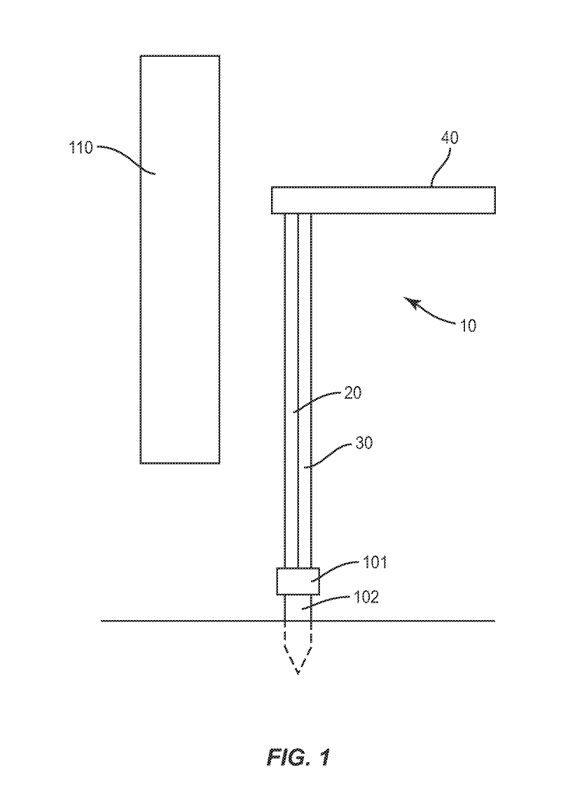

FIG. 1 schematically illustrates a device 10 designed to engage with an anchor 100. The device 10 includes first and second members 20, 30 that are movable relative to each other. The members 20, 30 are sized for the distal ends to be inserted into a head 101 that is pivotally attached to a shaft 102 of an anchor 100. Manipulation of a handle 40 that is operatively connected to one or both members 20, 30 moves the members relative to each other to expand a size of the first member 20 to engage with the head 101. The engagement leaves the exterior of the head 101 exposed for attaching an instrument. One or both members 20, 30 may further be shaped to facilitate attachment of the instrument 110 to the head 101.

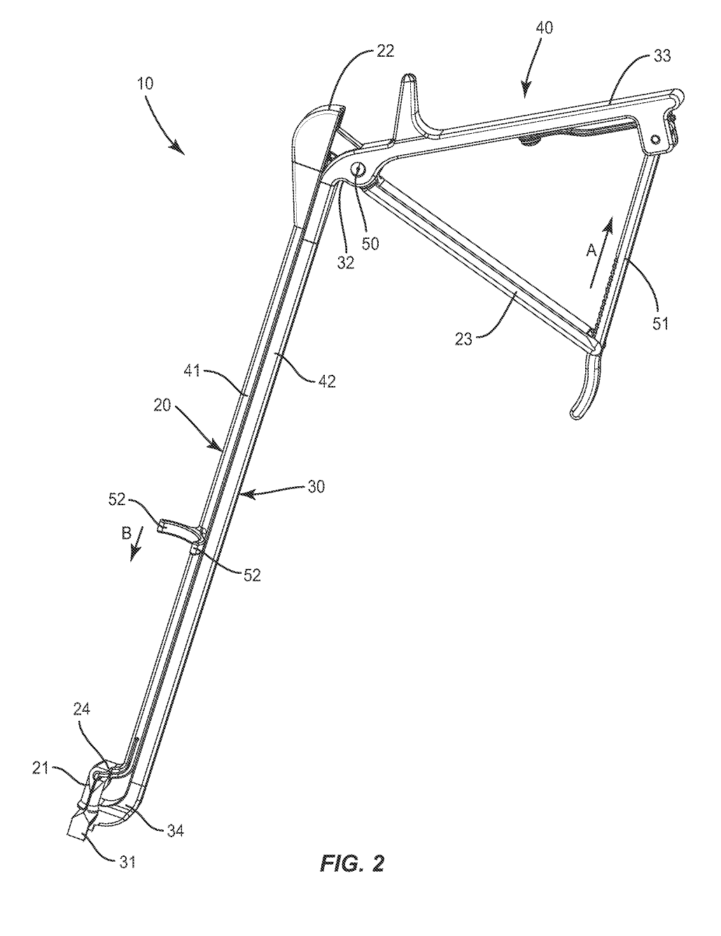

FIG. 2 illustrates a device 10 with first and second members 20, 30 that are movably attached together. The first member 20 includes an elongated shape with an elongated section 41 that is substantially straight and a collet section 21 at a distal end. The collet section 21 is sized and configured to fit within and expand to engage the anchor head 101. A bent section 24 is positioned between the elongated section 41 and the distal end. The bent section 24 positions the distal end radially outward away from a longitudinal axis of the elongated section 41. The elongated section 41 may be longer than both of the bent section 24 and the collet section 21.

The second member 30 likewise includes an elongated shape with an elongated section 42 that is substantially straight and a distal end having a base 31 sized to fit within the interior of the head 101 and to contact with and expand the collet section 21. A bent section 34 is positioned between the elongated section 42 and the base 31. The bent section 34 positions the base 31 radially outward away from a longitudinal axis of the elongated section 42. The elongated section 42 may be longer than the base 31 and the bent section 34.

A handle 40 is operatively connected to one or both members 20, 30 and is configured to cause relative movement between the first and second members 20, 30. The handle 40 may be formed by the proximal ends of one or both members 20, 30, or may be formed by additional elements attached to the members 20, 30. FIG. 2 includes the handle 40 formed by an extension 23 pivotally attached to a proximal end 22 of the first member 20. Handle 40 is also formed in part by a proximal section 33 of the second member 30. The proximal section 33 is offset from the remainder of the second member 30 by a bent section 32.

The elongated sections 41, 42 may abut together. Relative movement between the members 20, 30 causes the sections 41, 42 to slide along each other. The bent sections 24, 34 may be spaced apart and remain apart during the relative movement between the members 20, 30.

The first and second members 20, 30 are operatively connected together at a pivot connection 50. In a first orientation, the device 10 is sized to be inserted into the interior of the anchor head 101. Squeezing the sections 23, 33 together in the direction of arrow A causes the elongated section 41 of the first member 20 to move in a distal direction illustrated by arrow B along the elongated section 42 of the second member 30. This movement to the second orientation forces the collet section 21 over the base section 31 causing the collet section 21 to expand in width and engage with the anchor head 101. Movement of the sections 23, 33 apart back towards the first orientation causes the first member 20 to move in a proximal direction (i.e., opposite to arrow B) relative to the second member 30 resulting in the collet section 21 moving away from the base section 31. This proximal movement causes the collet section 21 to contract and disengage from the anchor head 101. A clamp 51 may extend between the ends of the handle 40 to maintain the sections 23, 33 at the desired position. A biasing member (not illustrated) may bias the sections 23, 33 apart to maintain the members 20, 30 towards the first orientation.

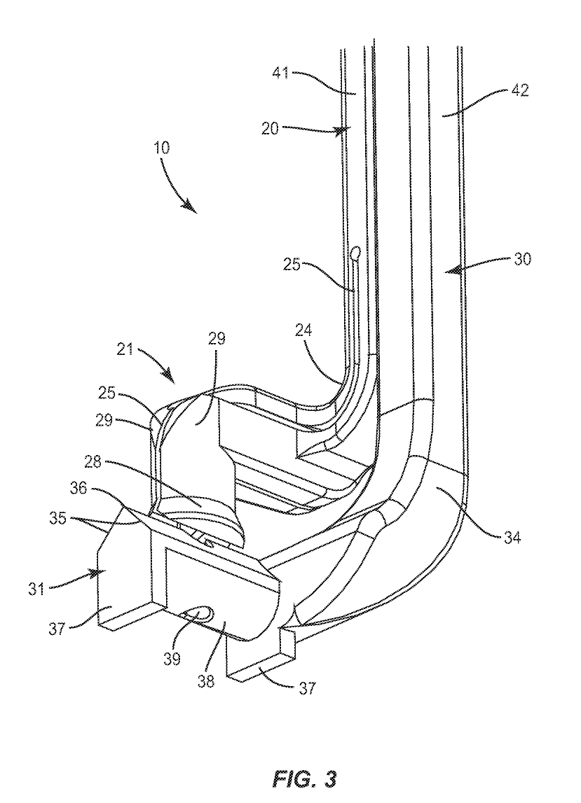

FIG. 3 illustrates the distal end of the device 10. The collet section 21 is positioned at the distal end of the first member 20 and is configured to expand within the anchor head 101. The collet section 21 includes a longitudinal slot 25 that extends inward from the distal end of the first member 20. As illustrated in FIG. 3, the slot 25 extends through the bent section 24 and terminates in the elongated section 41. The slot 25 may have other lengths, including terminating in the bent section 24, and terminating prior to the bent section 24.

The slot 25 divides the first member 20 into first and second longitudinal portions 29. In one embodiment, the slot 25 extends along a longitudinal centerline of the first member 20 with the portions 29 being equal in width. Other embodiments may include the portions 29 having different widths.

A V-gap 26 is formed in the slot 25 at the distal end of the first member 20. The V-gap 26 is formed by a first surface formed in the first portion 29 and a second surface formed in the second portion 29. The angles of the surfaces relative to the slot 25 may be the same or different. The V-gap 26 is sized to receive the base 31 of the second member 30.

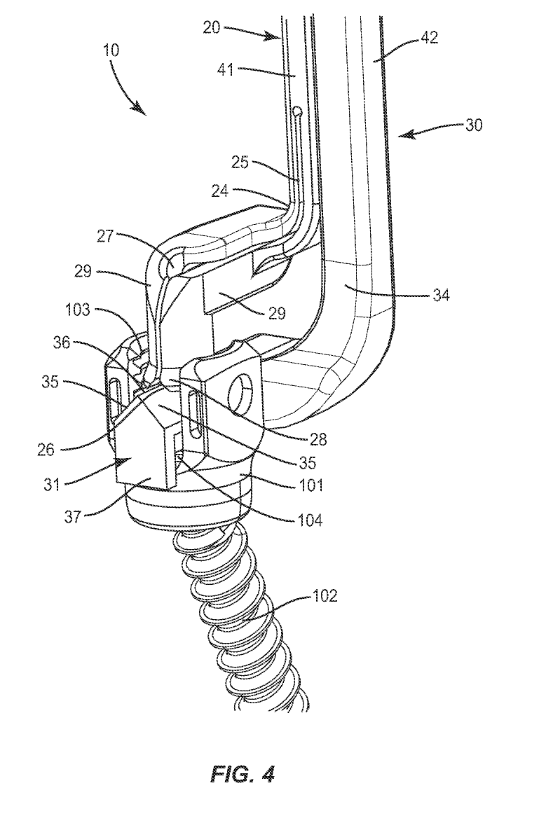

The collet section 21 may further include a flange 28 that extends outward away from the slot 25 to engage with the anchor head 101. The flange 28 may extend around an entirety or a limited periphery of the collet section 21. The flange 28 may be shaped and sized to engage with threads 103 on the anchor head 101. The flange 28 may be positioned at various locations along the collet section 21, with one placement being at the distal end as illustrated in FIGS. 3 and 4.

An aperture 27 may extend through the collet section 21 at the distal end of the first member 20. The aperture 27 may be formed at the slot 25 as best illustrated in FIG. 4, or may be positioned away from the slot 25. The aperture 27 is sized to receive a guide wire to position the device 10 within the patient. The embodiment of FIG. 4 includes the aperture 27 extending through a lower portion of the collet section 21 and through the distal end.

The second member 30 includes the base 31 located at the distal end. The second member 30 is sized and configured for the base to be located beyond the distal end of the first member 20. FIGS. 3 and 4 illustrate this positioning with the base 31 positioned below the collet section 21. The base 31 includes a central section 38 sized to fit within the interior of the anchor head 101. The central section 38 includes a first side that faces towards the first member 20 and includes angled surfaces 35 that form a peak 36. The surfaces 35 angle outward away from the first member 20 with a width of the central section 38 increasing away from the peak 36. The surfaces 35 may be oriented at the same or different angles. The base 31 is located relative to the first member 20 so the peak 36 is aligned with the slot 25.

The opposing second side of the central section 38 that faces away from the first member 20 includes a curved surface. The curved surface is shaped to correspond to a bottom of the channel 104 in the anchor head 101. Side walls 37 may extend outward on opposing ends of the central section 38 and extend outward beyond the curved surface. The side walls 37 are configured to be positioned on the exterior of the head 101 and straddle the channel 104. FIG. 4 illustrates the positioning with a first side wall 37 positioned at a first end of the channel 104 and a second side wall 37 positioned at a second end of the channel 104.

An aperture 39 extends through the central section 38 and aligns with the corresponding aperture 27 in the first member 20. The combined apertures 39, 27 form a passage to receive a guide wire to position the device 10 relative to the anchor 100. The passage may be parallel with the elongated sections 41, 42 of the first and second members 20, 30.

As illustrated in FIG. 4, the members 20, 30 are positioned to engage the anchor head 101 with a large majority of the exterior of the head 101 exposed for attachment of an instrument 110.

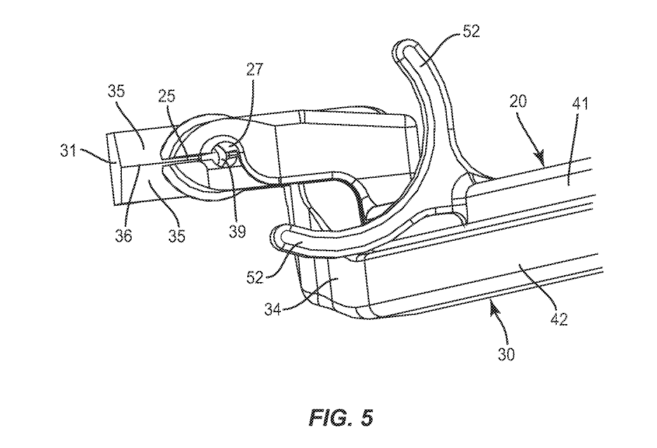

FIG. 5 illustrates the aperture 27 in the first member 20 that aligns with the aperture 39 in the second member 30. The combined apertures 27, 39 form a passage sized to receive a guide wire for positioning the device 10 within the patient. One or both members 20, 30 may further include a retainer 52 that extend laterally outward from the members 20, 30. The retainer 52 is sized to extend around a portion or entirety of an instrument 110 to position the instrument 110 relative to the device 10.

The device 10 is movable between the first orientation with the collet section 21 of the first member 20 having a first width for insertion into the anchor 101, and the second position with the collet 21 having an enlarged width for the collet section 21 to engage with the anchor head 101. In the first orientation, the distal end of the first member 20 may be spaced away from the base 31. Alternatively, the distal end may be in contact with the base 31 but the slot 25 not yet expanded such that the width allows for insertion of the collet section 21 into the anchor head 101.

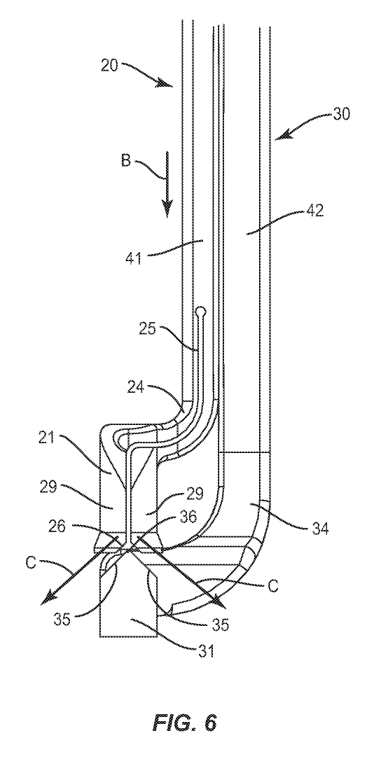

FIG. 6 illustrates the interaction between the distal sections of the first and second members 20, 30. The relative movement of the members 20, 30 is controlled by the handle 40 as discussed above. In one embodiment, compression of the handle 40 (not illustrated in FIG. 6) causes the elongated section 41 of the first member 20 to slide along the elongated section 42 of the second member 30 as indicated by arrow B. This movement further causes the collet section 21 of the first member 20 to engage with the base 31 of the second member 30. Specifically, the first portion 29 of the collet section 21 rides along the first angled surface 35 and the second portion 29 rides along the opposing second angled surface 35. The increasing width of the base 31 causes the portions 29 to move radially outward and separate as illustrated by arrows C. The outward movement of the two portions 29 increases the overall width of the collet section 21. The increase in width causes the outer surfaces of the collet section 21 to engage with the inner surfaces of the anchor head 101.

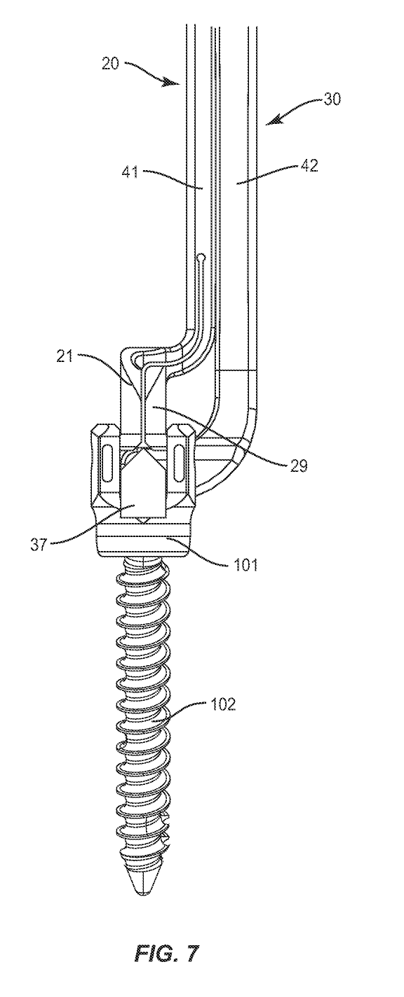

FIG. 7 illustrates the collet section 21 in an expanded orientation with the outer surfaces engaged with the anchor head 101. Once engaged, the handle 40 can be manipulated to position the orientation of the anchor head 101. The length of the members 20, 30 provides for the device 10 to be used in a percutaneous manner with the handle 40 positioned beyond the patient. Further, the attachment of the device 10 within the anchor head 101 leaves the exterior of the anchor head 101 exposed for attaching an instrument 110.

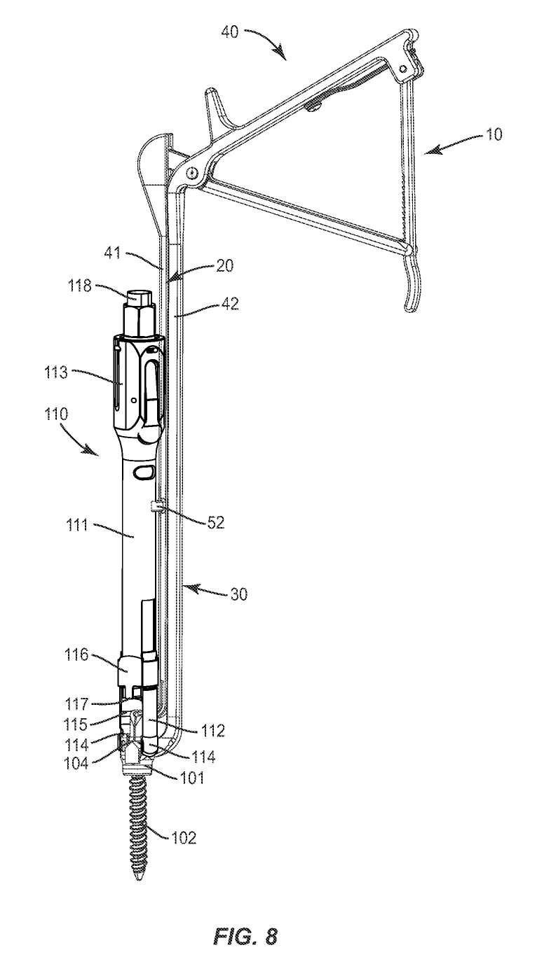

FIG. 8 illustrates an instrument 110 attached to the exterior of the anchor head 101 while the device 10 is attached to the anchor head 101. A variety of different instruments 110 may be attached to the anchor 100. One instrument 110 includes a hollow tube for accessing the anchor head 101.

FIG. 8 includes the instrument 110 being an extender with a tubular body 111 having a distal end 112 and a proximal end 113. The tubular body 111 includes a length such that the proximal end 113 extends outward from the patient when the distal end 112 is mounted to the anchor 100. The distal end 113 includes a pair of opposing legs 114 that connect to the exposed exterior of the anchor head 101. The legs 114 form an opening that aligns with the channel 104 to form a window 115. The distal end 112 may further include threads adapted for threading engagement with a corresponding portion of the anchor 100, or to an element coupled to the anchors 100. A sliding member 116 is movably positioned on the exterior of the tubular body 111 and located in proximity to the distal end 112. The sliding member 116 includes contact edges 117 that form an upper edge of the window 115. The proximal end 113 includes a fitting 118 that is operatively connected to the sliding member 116. Rotation of the fitting 118 in first and second directions causes the sliding member 116 to move downward and upward respectively along the tubular body 111 to position an elongated member in the channel 104. One example of an extender 110 is the Sextant Perc Trauma Extender available from Medtronic, Inc. of Minneapolis, Minn.

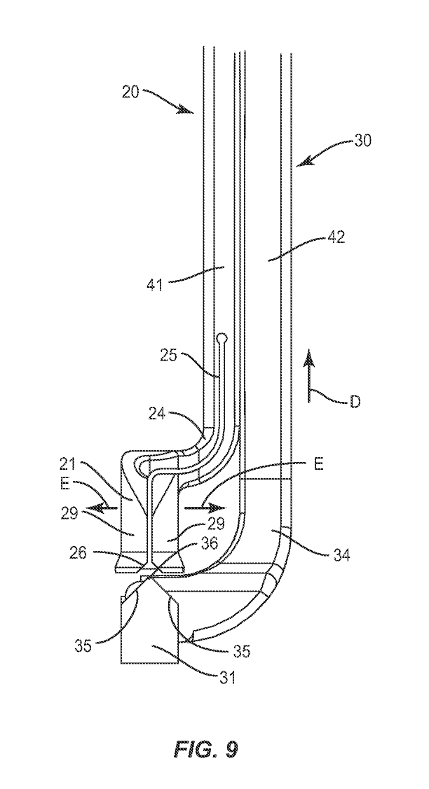

In one embodiment described above, compression of the handle 40 causes the elongated section 41 of the first member 20 to slide along the elongated section 42 of the second member 30. This movement is indicated by arrow B in FIG. 6 in which the collet section 21 of the first member 20 engages with the base 31 of the second member 30. The handle 40 may also be configured for compression to cause the elongated section 42 of the second member 30 to slide along the elongated section 41 of the first member 20 (i.e., the second member is the mobile member). FIG. 9 illustrates this configuration with the second member 30 moving in the direction of arrow D. The movement causes the elongated section 42 to slide along elongated section 41, and for the base 31 to move into the collet section 21 and move apart the portions 29 as illustrated by arrows E.

In the various embodiments, expansion of the collet section 21 is caused by relative movement between the members 20, 30. This may include one of the first and second sections 20, 30 remaining stationary and movement of the other section 20, 30. Embodiments may also include movement of both the first and second sections 20, 30.

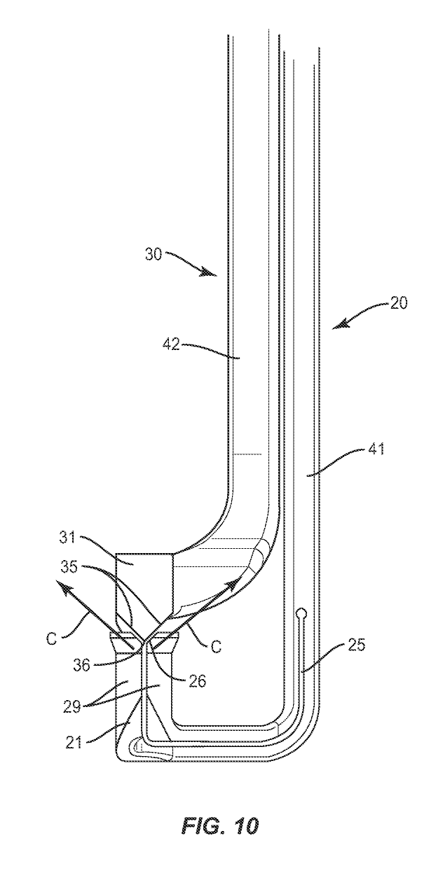

The embodiments described above, such as that illustrated in FIG. 6, include the base 30 positioned outward below the collet section 21. The device 10 may also include an arrangement with the collet section 21 positioned outward below the base 30 as illustrated in FIG. 10. In one embodiment, the first member 20 moves relative to the second member 30 to cause the collet section 21 to open as indicated by arrows C. Specifically, movement of the handle 40 causes the collet section 21 of the first member 20 to engage with the base 31 of the second member 30. The first portion 29 of the collet section 21 rides along the first angled surface 35 and the second portion 29 rides along the opposing second angled surface 35. The increasing width of the base 31 causes the portions 29 to move radially outward and separate as illustrated by arrows C. The outward movement of the two portions 29 increases the overall width of the collet section 21. The increase in width causes the outer surfaces of the collet section 21 to engage with the inner surfaces of the anchor head 101. The embodiment of FIG. 10 with the collet section 21 positioned outward below the base 30 may also include a handle 40 configured for the second member 30 to be mobile and move relative to the first member, or for both members 20, 30 to be mobile.

The device 10 may be used in a variety of different manners during various surgical procedures. One procedure includes attaching the device 10 to the anchor 100 prior to the anchor 110 being inserted into the patient. Other methods include attaching the device 10 to the anchor 100 after the anchor 100 has been inserted into the patient and the shaft 102 attached to a bone or tissue. The elongated shape of the device 10 provides for use in minimally invasive procedures as just a limited section of the device 10 is inserted into the patient with the remainder remaining outside of the patient.

In the various procedures, the collect section 21 and base 31 are positioned within the interior of the anchor head 101 while the device 10 is in the first orientation. The device 10 is then manipulated to expand the collet section 21 as the device 10 is moved to the second orientation. Once engaged, the device 10 can be manipulated to position the anchor head 101 at the desired orientation within the patient.

An instrument 110 may then be inserted into the patient and attached to the exposed exterior of the anchor head 101. The instrument 110 may be inserted within the same incision as the device 10 and may be attached to the device 10 either during or after insertion into the patient. The device 10 may also be used in procedures that do not include attaching an instrument 110 to the anchor head 101.

In the various procedures, the device 10 is positioned within the interior of the anchor head 101 and therefore is removed prior to insertion of the elongated member to free the channel 104 to receive the elongated member. Detachment may occur at various times during the procedures.

The device 10 may be used for positioning a variety of anchors 100 within the patient. One type of anchor 100 is a multi-axial anchor that includes a head 101 that is movably attached to a shaft 102. The shaft 102 may include threads, a hook, or other structural aspects to engage with a bone within the patient. The head 101 includes the channel 104 to receive the elongated member. The anchor 100 includes a set screw sized to fit over the channel 104 and engage with threads 103 to secure the elongated member within the channel 104. Examples include CD HORIZON and VERTEX anchors, each available from Medtronic, Inc. of Minneapolis, Minn. The device 10 may also be used with mono-axial anchors that include a fixed head 101 on the shaft 102. The device 10 may also be used for various other types of anchors that are used during various surgical procedures.

FIG. 2 includes the members 20, 30 having a bent shape. This shape provides for the elongated sections 41, 42 to be laterally offset from the anchor 100. This shape provides for an instrument 110 such as an extender to be inserted along the sections 41, 42 and onto the anchor 100. In another embodiment, the members 20, 30 have a substantially straight shape. The member 20, 30 may be arranged in a telescoping configuration with one member over the other. The members 20, 30 may also be configured for the members to be inter-twined together. In one embodiment, the members 20, 30 are connected together by a threaded collar. Rotation of the collar moves one of the members 20, 30 relative to the other to engage and disengage the anchor 100. The straight shape of the members 20, 30 provides for insertion directly over and onto the anchor 100. Once attached, an instrument 110 such as an extender may be inserted over the members 20, 30 and slid downward into engagement with the anchor 100.

The instrument 10 may be used during surgical procedures on living patients. The instrument 10 may also be used in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A device to attach to an anchor during a surgical procedure, the device comprising:
elongated first and second members movably connected together for movement of the first member relative to the second member, each of the members including a distal end and a proximal end;
a collet section at a distal end of the first member, the collet section including a longitudinal slot that that extends inward from the distal end and forms a longitudinal first portion on a first side of the slot and a longitudinal second portion on an opposing second side of the slot;
a base at the distal end of the second member, the base including opposing first and second angled surfaces that form a peak, the base positioned with the peak being aligned with the slot in the collet section;
the first member being movable towards the second member to separate the collet section at the slot with the first portion configured to move along the first angled surface and move outward in a first lateral direction and the second portion configured to move along the second angled surface and move outward in a second lateral direction.

2. The device of claim 1, wherein the first member includes a straight section and a bent section, the straight section having a greater length than the collet section and the bent section, the bent section positioning the distal end radially away from a longitudinal axis of the straight section.

3. The device of claim 2, wherein the slot extends inward from the distal end through the bent section.

4. The device of claim 1, further comprising a flange that extends radially outward from an exterior of the collet section and is configured to engage the anchor when the first and second portions move laterally outward.

5. The device of claim 2, further comprising a first aperture that extends through the collet section and a second aperture that extends through the base, the first and second apertures being aligned to form a passage that is parallel with the longitudinal axis of the straight section.

6. The device of claim 1, further comprising a V-shaped gap positioned at the distal end of the first member at the slot.

7. A device to attach to an anchor during a surgical procedure, the device comprising:
a first member having an elongated straight first section, a first bent section that bends away from a longitudinal axis of the straight first section, and a first distal end on an opposing side of the first bent section from the straight first section, the straight first section being longer than the first bent section;
a second member having an elongated straight second section, a second bent section that bends away from a longitudinal axis of the straight second section, and a second distal end on an opposing side of the second bent section from the straight second section, the straight second section being longer than the second bent section, the second distal end being positioned beyond the first distal end of the first member;
a connection that connects together the first and second members with the first and second sections being aligned together;
a longitudinal slot in the first member that extends inward from the first distal end, the slot dividing the first member into a first portion on a first side of the slot and a second portion on a second side of the slot;
a base at the second distal end including a first side that faces towards the first distal end and includes a pair of ramped surfaces and an opposing second side configured to seat within the anchor;
the first member being movable relative to the second member between a first longitudinal orientation with the first distal end having a first width and a second longitudinal orientation with the first distal end overlapping the base to separate the first and second portions a greater amount with the first distal end having a larger second width.

8. The device of claim 7, wherein the slot extends through the first bent section of the first member.

9. The device of claim 7, wherein the first and second straight sections abut together and the first and second bent sections are spaced apart.

10. The device of claim 7, further comprising a handle operatively connected to each of the first and second members, the handle configured to move the members between the first and second orientations.

11. The device of claim 7, wherein the ramped surfaces join at a peak that extends along the base and aligns with the slot in the first member.

12. The device of claim 7, further comprising a first aperture that extends through a length of the first member at the first distal end, and a second aperture that extends through the base of the second member, the first and second apertures being aligned together to form a passage that is parallel with the straight first and second sections.

13. The device 7, wherein the first distal end overlaps with the base in the first orientation.

14. A method of positioning an anchor within a patient during a surgical procedure comprising:
positioning a collet section of an elongated first member and a base of an elongated second member in a channel in a head of an anchor with the base at a bottom of the channel and the collet section above the base and away from the bottom, the collet section including first and second portions separated by a longitudinal slot that extends longitudinally inward from a distal end of the first member, and the base includes a first side that faces towards the collet section with first and second ramped surfaces that form a peak that increases in width away from the collet section;
moving the collet section relative to the base towards the bottom of the channel;
moving the first portion of the collet section along the first ramped surface of the base and moving the second portion of the collet section along the second ramped surface of the base;
increasing a width of the collet section as the first and second portions move along the ramped surfaces; and
engaging outer surfaces of the first and second portions with the anchor head that forms the channel and having an exterior of the anchor head being exposed.

15. The method of claim 14, further comprising positioning a curved section of a second side of the base against the bottom of the channel and straddling first and second sidewalls that extend outward beyond the curved section on opposing sides of the anchor head.

16. The method of claim 14, further comprising sliding an elongated first section of the first member along an elongated second section of the second member.

17. The method of claim 14, further comprising moving the collet section relative to the base away from the bottom of the channel and disengaging the outer surfaces of the first and second portions with the anchor head.

18. The method of claim 14, further comprising positioning bent sections of the first and second members away from the channel while positioning the collet section of the first member and the base of the second member in the channel in the head of the anchor.

19. The method of claim 14, further comprising attaching an instrument to the exterior of the anchor head while the outer surfaces of the first and second portions engage with the anchor head.

20. The method of claim 14, further comprising moving the device along a guide wire that extends in the anchor with the guide wire extending through apertures that are aligned in the distal ends of the first and second members.
